(19) United States
(12) Patent Application Publication
(10) Pub. No.: US 2025/0262413 A1
(43) Pub. Date: Aug. 21, 2025

(54) non kink IV device for placement on a patients arm called a Silent antecubital or Silent AC.

(71) Applicant: David Whirls, JR., Middletown, IN (US)

(72) Inventor: David Whirls, JR., Middletown, IN (US)

(21) Appl. No.: 19/052,890

(22) Filed: Feb. 13, 2025

Related U.S. Application Data
(60) Provisional application No. 63/553,988, filed on Feb. 15, 2024.

(51) Int. Cl. A61M 25/02; A61M 25/00

(52) U.S. Cl. CPC A61M 25/02; A61M 2025/0059; A61M 2025/0213; A61M 2025/0246; A61M 2025/0253

(57) ABSTRACT

This is a non-kink IV device for placement on an arm of a patient called a Silent antecubital (AC). The non-kink IV device includes a small amount of a compressed material with a rounded bottom, at least one elastic band, and a stretchy net piece to encase the roll and bands around the arm of the patient. The device holds the IV against the arm so that arm movement of the patient is limited to avoid kinking of any tubing for the IV since the kinking can set off an alarm. Options for the compressed material are a compressed roll of gauze, an inflatable bladder, a soft rubber ball which can be a soft rubber made of a fully FDA compliant material such as rubber, silicone, various plastics, a composite material, and a fiber cloth.

### CROSS-REFERENCE TO RELATED APPLICATIONS

This application claims the benefit of U.S. Provisional Patent Application with Ser. No. 63/553,988 filed Feb. 15, 2023, by David Whirls. The provisional application is entitled “A non-kink IV device for placement on a patient's arm called a Silent antecubital or Silent AC.”

### FIELD OF INVENTION

This invention relates to a non-kink IV device for placement on a patient's arm called a Silent antecubital or Silent AC. The present invention relates to a device for protecting an intravenous site of a patient from kinking and then setting off an alarm from a no-flow situation. Particularly, the embodiments described herein relate to devices and methods for transferring fluid to or from a patient through a placed peripheral intravenous catheter, The present invention generally relates to means of securing an intravenous catheter and tubing in use with a patient, and more particularly related to a device for securing and stabilizing an intravenous catheter and associated tubing adjacent to the site of entry of the catheter through the skin of the patient, protecting the catheter and its entry site from interference, and facilitating access to the catheter for maintenance by medical personnel while minimizing physical trauma to the patient.

### FEDERALLY SPONSORED RESEARCH

None.

### SEQUENCE LISTING OR PROGRAM

None.

### BACKGROUND—FIELD OF INVENTION and PRIOR ART

As far as known, there are no other non-kink IV device for placement on a patient's arm such as the Silent antecubital or Silent AC. One of the major benchmarks of medical care was the introduction of an Intravenous (IV) set to access the circulatory system of a patient, enabling the administration of fluids and medications in a controlled, predictable manner. The typical IV set includes a primary fluid flow line of tubular construction with one or more access points. Some of these access points can comprise access ports that allow the administration of medications through either a syringe by push or by infusion through another IV set (primary or secondary). A primary access point is located at one end of the IV set with a fluid source, such as normal saline or some other carrier fluid. A spike and drip chamber assembly are positioned at a terminus or proximal end of the flow line with means for attachment to a patient injection site on a distal end. Secondary flow lines may be combined with the primary flow line with similar construction options.

Use of IV sets has now become ubiquitous at every stage of medical care, from the site of an accident or injury, through transport to the hospital, during emergency room and surgical procedures and potentially continuing into the ICU and general hospital care. At each successive stage of procedure or treatment, different medical personnel typically become involved. Normally, later stage medical personnel will not have actual firsthand experience with a patient and the various multiple attached IV sets. Often, they may have to guess as to the purpose and procedure associated with each previous IV set, including what medications may have been administered. As medical technology becomes advanced, it becomes increasingly apparent that the care of patients after surgery or in advanced stages of illness is quite sensitive and requires the imposition of different parameters for each individual patient. For example, after surgery, each individual patient requires the introduction of different amounts of fluid at preselected rates in accordance with a number of parameters unique to each patient.

Intravenous infusion of fluids and intravenous removal of fluids has been and continues to be a common practice in the medical treatment and care of patients in hospitals and other medical facilities. A typical intravenous infusion system comprises a catheter penetrating the skin and an underlying vein of, most commonly, the patient's arm, a source of fluid, and tubing interconnecting the source of fluid and the infusion catheter. To minimize movement of the needle or catheter relative to the limb of the patient and to prevent inadvertent removal of the catheter, it is standard practice to secure the catheter and a portion of its associated tubing to the limb of the patient. With conventional methods of medical practice, a needle-bearing catheter is inserted through the skin of the patient into an underlying vein, the needle is removed, and the catheter is secured to the skin of the patient with adhesive tape. In addition to securing the catheter, a portion of the associated tubing is looped or coiled and similarly secured to the patient's skin with several strips of adhesive tape for the purpose of absorbing any tension imposed upon such tubing without displacing the catheter. While this conventional system has proven to be reasonably effective in securing the catheter and tubing, it has several disadvantages.

### Problems Solved

Many problems and irritations are solved with the advent of the non-kink IV device for placement on a patient's arm called a Silent antecubital or Silent AC. The advantages help the Patients, nurses, hospital and suppliers as will be understood by reviewing the advantages, below. There is a need for an IV device to eliminate the kinking of the tubing which shuts down the flow of fluids and medication as well as setting off an alarm to the nursing staff to correct the no-flow.

### Prior Art

A novelty search was completed for a non-kink IV device for placement on a patient's arm called a Silent antecubital or Silent AC. It revealed the following:
A. AU2009345108—by Khair et al., in 2009 for System and Method for delivering and monitoring medication. A method of delivering medication to a patient utilizing a medication delivery system is provided. A first medication to be delivered to the patient is supplied. The patient's identity is verified. A visual display provides information related to the injection. The method updates the patient's electronic medical administration record to capture the information regarding the injection of the first medication
B. U.S. Pat. No. 5,112,313 by Sallee in 1992 for an IV Cover/Protector. The present invention is directed to an intravenous needle protector which comprises a housing with a body and a terminating surface, at least one opening is in the body at the terminating surface for passage of the tubing which extends from the intravenous needle. The terminating surface extends from the body for the attachment of tape. The tape may be a part of the terminating surface or applied to that surface as well as to a person's body to hold the housing over the area of the intravenous needle injection. The body includes recedable retention means for retaining the needle.
C. U.S. Pat. No. 4,056,333 by Lundquist in 1977 for an Intravenous Feeding Pump Failure Alarm System. The present invention relates to a failure alarm system for an intravenous feeding pump in which the pumping force is secured by the projection of a plunger into a chamber filled with the liquid to be pumped, which plunger is encased in a tightly fitting sheath of elastic material. In such a pump, actuation is secured by the application of a force from an actuator to the end of the plunger extending without the wall of a pumping chamber and the return force is secured by the elasticity of the sheath. The actuator of such a pump provides an actuator which can be selectively set to provide an extended range of operations per unit of time and can be modified to control the amount of projection into the pumping chamber by the plunger. In such a combination means is provided by the present invention for sensing the fact that there is a negative pressure, or suction, in the system that is not balanced by the in-flow of liquid, i.e., the amount of fluid delivered to the pump for each operation is less than that required by the setting (thus sensing when a supply is shut off, or less than the preset amount of liquid is reaching the pump),
D. U.S. Pat. No. 4,391,599 by Jenkins in 1983 for an Apparatus for Providing a Controlled Flow of Intravenous Fluid to A Patient. Apparatus provides a controlled flow of fluid at particular rates from a source to a patient at controlled pressures of low value. The apparatus includes an input line connected to the source. An output line extends to the patient and implementing means such as a pump implements the flow of fluid from the input line to the output line at a particular rate. The source is disposed a particular distance above the implementing means to produce a particular pressure of the fluid at the implementing means and to provide this particular pressure to the fluid flowing to the patient.
E. U.S. Pat. No. 4,976,698 by Stokley in 1990 for an Intravenous Catheter and Tubing Stabilization Device. A device for securing and stabilizing an intravenous catheter and a portion of its associated tubing in place relative to the limb of a patient, comprising a planar base to be removably attached to the limb of a patient adjacent the catheter entry site, having a pair of U-shaped walls defining an accurate slot therebetween to receive and stabilize the catheter tubing, and further having a cover adapted to be removably interconnected to the planar base to retain and stabilize the catheter and associated tubing relative to the limb of the patient and prevent direct access to the catheter entry site. The device is preferably constructed of a clear plastic material and is preferably attached to the limb of the patient with one or more flexible adhesive strips.
F. U.S. Pat. No. 6,080,138 by Lemke et al., in 2000 for an IV Protector. The IV protector of the present invention is a device designed to prevent spattering of blood from the tip of a peripheral IV catheter during removal and disposal of the catheter and IV tubing. The IV protector is a flexible plastic cylinder about three inches in length and about 1.5 cm in diameter. At one end it has a projection and a mating groove clasp fastener in order to seal the end of the cylinder by pinching the projection into the groove. At the other end the cylinder has internal threads adapted to lock the IV protector in a retracted position circumferentially about the IV tubing when the catheter is inserted in the venipuncture site and adapted to lock the IV protector in an extended position circumferentially enclosing the catheter for disposal. A pair of resilient threaded washer shaped adaptors which snap fit on the IV tubing, or tubular adaptors having a hub adapted for connection to the catheter hub at one end and adapted for connection to the needle adaptor of the IV tubing at the other end are used to dispose the IV protector between the IV tubing and the catheter.
G. U.S. Pat. No. 8,088,090 by Felt et al., in 2012 for an Extracorporeal Blood Processing Methods with Multiple Alarm Levels. This invention provides a method for controlling a fluid separation system, preferably a blood apheresis system having a leukocyte reduction chamber. The method utilizes at least a two-level alarm system. A first-level alarm condition is triggered in response to a pressure drop in the system to less than or equal to a specified system pressure and pauses fluid flow in at least a portion of the system. A second-level alarm condition is triggered in response to a specified number of said pressure drops within a specified period and reduces the flow rate of the fluid in the system. The alarm conditions may also trigger a visible and/or audible alarm. These alarm levels permit flow through the leukocyte reduction chamber to continue when the pressure drop is caused by a non-serious, self-resolving or easily correctable condition such as misalignment of system components.
H. U.S. Pat. No. 10,463,783 by Reichert et al., in 2019 for an IV System with Bypass Manifold. An intravenous (IV) set system comprising a primary IV set defining a primary flow line of the IV set system. The primary flow line can include multiple access points along its length and feeds to a merging fluid pathway proximate to the distal terminus of the primary IV set. At least one secondary IV set with corresponding flow lines can be separably joined to the primary IV set to cause the secondary flow lines to be in fluid communication with the primary flow line. At least one of the IV sets can have a unique set of uniform marking indicia to facilitate rapid identification of the IV sets from other IV sets within the IV set system. Such marked and identified primary and/or secondary IV sets can be allocated to specific medical functions and/or can be for use by specific medical personnel to thereby minimize risk of error in administration of an IV to a patient. The IV set system can further comprise a merging fluid pathway about a primary IV set.
I. U.S. Pat. No. 11,090,461 by Ehrenreich et al., in 2021 for a Devices and Method for Fluid Transfer Through a Placed Peripheral Intravenous Catheter. An apparatus includes a catheter, an introducer, and an actuator. A distal end portion of the introducer is configured to couple to an indwelling peripheral intravenous line. The actuator is movably coupled to the introducer and is configured to move the catheter between a first position, in which the catheter is disposed within the introducer, and a second position, in which a distal end portion of the catheter is distal to the introducer. A first portion of the actuator is disposed outside of the introducer and in contact with an outer surface of the introducer such that (1) a longitudinal axis defined by a second portion of the actuator is nonparallel to a longitudinal axis defined by the introducer and (2) the second portion of the actuator exerts a force on a proximal end portion of the catheter operable to increase an internal stress within a portion of the catheter.
J. U.S. Pat. No. 11,626,207 by Yang et al., 2023 in for a Method and Systems for Providing Customized Settings for Patient Monitors. A method for providing one or more customized alarm setting recommendations for a patient includes the steps of: providing a patient monitor configured to monitor the patient, the patient monitor comprising a patient sensor and a processor configured to receive the sensor data from the patient sensor, receiving, by the patient monitor, information about a patient; analyzing, by the processor using an alarm setting recommendation classifier, the received information about the patient to generate one or more alarm setting recommendations customized to the patient; providing the one or more alarm setting recommendations to the user, and receiving input from the user accepting, rejecting, and/or modifying the alarm setting recommendations.
K. US2005/0185799 by Bertram in 2005 for a Method of Monitoring Equipment and Alert Device. A device and method that interfaces between a medical device and a call alert system. The device includes a microphone that senses an audio signal generated by the medical device when the medical device alarms.
L. US2006/0247577 by Wright in 2006 for a Flexible IV Site Protector. A venipuncture site protector that includes a securement and a cover mounted to said securement. The cover includes a proximal end having a front wall with a tube receiving slot. The cover also includes a distal end having an arch that forms an opening that lies on a vertical plane that is substantially perpendicular to the securement.
M. US2020/0021930 by Iswanto et al., in 2020 for a Patient Monitor Alarm Speaker Analyzer. A patient monitor can diagnose whether its speaker is blocked, malfunctioning, or at a volume that is too low. For example, the monitor can include a processor that can diagnose the speaker by recording a microphone input signal. The processor can compare the microphone input signal to an expected alarm signal that should be output by the speaker. If the two do not match or reasonably correspond to one another, then the processor may increase the volume of the alarm to determine whether doing so can overcome an obstruction, noise, or potential malfunction. The microphone can again detect the speaker output, and the processor can again make another comparison or analysis of the input with the speaker output. If the speaker output as detected via the microphone is still insufficiently loud, then the patient monitor may output an indication that the speaker has a problem.
N. EP0185977A1 By brown and Henry Multilumen catheter set. A subcutaneous catheter set (100) is disclosed in which a multilumen catheter tube (10) has a tissue cuff (20) connected about its outer circumference. A lock adapter (40) (62) (64) has a plurality of conduits in communication with the lumens of the multilumen catheter tube (10). A complementary lock adapter is connected to another multilumen catheter tube, a plurality of single lumen lines, a plurality of injection tubes or may be plugged to act as a seal. The two lock adapters can be locked together so that the subcutaneous multilumen catheter may be connected directly to any of the items attached on the complementary lock adapter.
O. U.S. Pat. No. 9,561,319B2 By Bongers Device and method for fixing a segment of tubing, for an arrangement for monitoring an access to a patient. A device for fixing a segment of tubing in the form of a loop, for an apparatus by which a fluid is fed to or out from a patient via tubing, in particular for monitoring vascular access in extra-corporeal blood treatment, and a tubing and an arrangement for monitoring, each having the device for fixing the segment, are also described. The fixing of the segment includes a guiding piece and a securing piece. The guiding piece fixes intersecting portions of the segment which forms an eye, thereby forming a loop which contracts under a tractive stress. The securing piece fixes a portion of the segment forming the eye such that when the loop tightens, that portion of the segment does not slip through the guiding piece. Alternatively, intersecting portions of the segment are fixed in a loop only by a guiding piece formed as an annular body.

As can be observed, none of the prior art has anticipated or caused one skilled in the medical and medical care industry to see this unique Silent AC invention as obvious to one skilled in the industry. The non-kink IV device for placement on a patient's arm provides an answer to an IV application to avoid all the problems mentioned above as well as the advantages listed below in an efficient manner.

### SUMMARY OF THE INVENTION

This invention is a non-kink IV device for placement on a patient's arm called a Silent antecubital or Silent AC. The preferred embodiment of the Silent AC non-kink IV device is comprised of: A non-kink IV device called a Silent antecubital or Silent AC for placement on an intravenous (IV) device on a patient's arm comprised of: (a) a compressed material such as a roll of gauze with a rounded bottom; (b) At least one elastic band; and (c) a stretchy net piece to encase the roll and elastic bands Wherein the patient's Intravenous (IV) device is held and the arm movement of the patient is limited to avoid kinking the tubing and setting off an alarm. Other options of inflatable bladder/ball and inserting the roll into the stretchy netting are also included.

### OBJECTS AND ADVANTAGES

There are several objects and advantages of the non-kink IV device for placement on a patient's arm called a Silent antecubital or Silent AC. These various advantages and benefits that help the Patients, nurses, hospital and suppliers include:

### A. Advantages to Patients

1) Better quality sleep for the patient and better overall mood.
2) More likely hood to fill out customer service ratings in a positive light due to having had a better experience.
3) As a patient who hates needles (as most people do) one would not be okay getting a second IV done for no medical reason to one's health such as if due to an alarm annoyance or nurse time it would not be okay with that added risk to a patient.
4) Noise levels are already loud and a room for the patient is already a busy place, alarms from other patients or their rooms is an extra layer of noise.
5) Feeling that the care for a patient is important to the staff, if the is an annoyance due to an alarm for accidentally being human and bending ones arm it causes tense interactions to form between staff and the patients causing patients more likely to feel less cared about being treated poorly.
6) The ability to eat unrestricted and staying on schedule for timed medications due to unrestricted IV Pump infusions.
7) This helps allow ad lib patients access to move the IV Pole to the bathroom themselves when needed. Without the new non-kink device, the alarm may sound when the patient is moving to the bathroom and back.
8) Helps promote ambulation for a patient in the halls with less fear of setting off an alarm while manipulating an IV Pole during ambulation thus promoting better health.
9) Less risk of infection from a prevented 2nd IV need of a moved IV placement.

### B. Advantages to Nurses

1) Time saver. Some registered nurses (RNs) will spend many hours of their shift repeatedly going into the room of an alarming patient to restart IV pumps.
2) The RN becomes frustrated over the repeated interruption to their care elsewhere. This frustration is on both the patient and the nurse leading to tension built between both. Because the RN is constantly having to reset the alarm and constantly reminds the patient to unbend their arm. This becomes a power struggle between the patient and nurse with the constant reprimanding.
3) The current method of preventing this situation (in a confused patient for example) is an Arm Board. It is literally where the nurse straps a padded board to the forearm and bicep area. This completely prevents the patient from moving their arm. This form of restraint is almost a barbaric practice.
4) The current solution with the Arm Board often causes other problems to arise. In most situations the Arm Board is secured to the arm with a product commonly known as Coban. This Coban product often gets applied too tightly (the more the patient moves their arm the tighter the Coban restricts) and this eventually leads to restricted blood flow of the arm. This is a restriction which could lead to host of other medical issues. For example, in the worst-case scenario it has caused DVT (life threatening blood clot with potential to cause a stroke) to form in the patient's arm.
5) The only other solution to this issue with the alarm is to place a new IV elsewhere on the patient's arm. This itself has its own challenges. Most patients do not like the idea of having a new IV placed simply because of a mechanical hardware issue. Needles cause some patients extreme anxiety. Also, the need for another IV will cost the hospital significantly more money in supplies and nursing time.
6) Some patients are considered “Hard Sticks.” Meaning they have poor veins from which to select an IV placement. Typically, in this type of patient the antecubital (AC) is the best and easiest vein to get a successful IV placement completed. Also, an IV placed in the AC area is most likely to give good blood return in the future. This gives the nurse blood access for Lab work in the future for that “hard stick” patient.
7) Patients still need to be able to feed themselves while in the hospital. A patient with a positional IV in the AC will likely trigger the pump alarm while eating since it is nearly impossible to feed oneself without bending the arm at the elbow. The current solution is to shut down the IV pump while the patient eats to avoid triggering the alarm repeatedly while the patient eats. Applying the Silent AC will simply prevent the alarm from triggering while allowing the patient to feed themselves. This saves the registered nurse (RN) time and prevents rescheduling of timed medication infusions. Ultimately this provides a better plan of care for the patient.
8) Nurses want happy patients that are willing to go along with their intended medical plan of care. Power struggles over constant alarms being triggered simply because the patient forgot to not bend their arm leads to these power struggles. These power struggles do not promote happy patients. Everyone involved would prefer that the patient is well rested and happy with their medical care and not frustrated with a power struggle over an alarming IV pump.
9) The stretch netting that will be in the Silent AC kit has other good uses. It can be used to help redirect the IV tubing away from the insertion site and also help to prevent a kink from developing in the IV tubing by redirecting the tubing away from the site in a such a manner that keeps the IV tubing controlled and not bending over itself. This diversion helps prevent another area from developing a kink in the system.

### C. Advantages to Hospitals

1) Saves time for the Nurses. This Silent AC device helps to keep the nurse doing more productive things with their time since they are not distracted from alarming pumps. This leads to better time management.
2) The Silent AC reduces alarm fatigue which means a nurse will be more likely to actually respond to the alarms in a timelier manor.
3) The Silent AC will lead to an overall quieter and more peaceful hospital environment by reducing the total amount of alarm noises. This calmer environment will promote better health. Remember that IV pump alarms are not just heard by the affected patient and nurse but everyone else on that hospital unit.
4) Patient satisfaction ratings are a big part of hospital culture these days. As a hospital management team ask oneself this question. Does one want the customers (patients) to fill out a survey about their satisfaction with their hospital experience after having a good peaceful night's rest? Or rather after an IV pump woke them up countless times through the night. Ending their night more exhausted than when they attempted to fall asleep. Because when people fall asleep it is only natural that the arm will bend as we all revert to a more fetal like position in our sleep. The Silent AC helps to prevent the IV pump alarm from ever triggering due to a kink in the line caused by the bending of the arm.
5) Cost Savings! There are only a couple of solutions to the alarming pump issue today. One solution is that a nurse can now place a new IV. The design of the Silent AC helps to prevent the IV pump alarm from ever triggering due to a kink in the line caused by the bending of the arm. If at the antecubital, that helps to prevent the IV pump alarm from ever triggering due to a kink in the line caused by the bending of the arm. A new IV at the AC location requires the following: a new IV, another IV start kit, a band aid for the old IV site, and yet another chance for something to go wrong during another new IV start (like an infiltrated IV or a number of negative things associated with an unsuccessful IV start attempt). The second solution is the use of a padded arm board. This has the cost of the arm board itself as well as the cost of the wrapping (Coban).
6) Probably the biggest cost saver for the hospital is the 15-30 minutes of Registered Nurse (RN) time required to place a new IV in a best-case scenario. The RN time is extremely valuable in the hospital setting and their time is better spent on other issues. This waste of time could be avoided with the use of a relatively inexpensive device (the new Silent AC) preventing the IV pump alarm situation and saving the original otherwise completely functional IV.
7) It is only natural for a Registered Nurse (RN) in an emergency room environment to place an IV in the AC location because it is normally the easiest place to get a successful IV on the first attempt. It is the same for a paramedic in an ambulance situation. They do not consider that later this IV placement location may cause an alarm to get triggered when an IV pump is used to administer timed medications. The emergency room nurse and the paramedic just need immediate IV access. Especially in an emergency situation. The possibility that the IV location in the AC will later cause an alarm issue with a pump is not even considered in the moment.
8) Overall, less IV related issues will develop for the hospital associated with un-successful IV starts. Examples of these are hematomas, bruises, and infiltrations at the IV site. All of these have the potential to lead to even more serious risks and can be avoided by placing the Silent AC on the original IV placed in the emergency situation.

### D. Advantages to Suppliers

1) The added opportunity to sell precut lengths of stretch netting included in the Silent AC kit. The precut would have a much higher profit margin for the precut lengths of stretch netting.
2) The possibility of adding this new device to a packaged IV start Kit intended for starting an IV in the AC location. (This is the first location most nurses and paramedics will look for in a patient).
3) An opportunity for the educational promotion of the product. This increases opportunities for medical sales teams. This could be an introduction or foot in the door with a hospital that does not currently have a solution to the IV pump alarm issue. This introduction could lead to future sales of other different medical supplies for this manufacturer.
4) The ability to package this product in several different ways.
   a. Individually just the Silent AC without stretch netting for those hospitals that already stock rolls of stretch netting in various sizes.
   b. As a kit that includes precut lengths of stretch netting along with the Silent AC for situations where the IV is already placed in the AC location and now causing an alarm issue with the IV pump.
   c. Packaged as part of an IV Starter Kit intended for IV starts in the AC location. (This is the most common location for an IV to be placed).
5) All the above situations lead to increased total sales and better customer satisfaction.
6) The Silent AC will only cost pennies per unit to manufacture and will have the ability to sell for several dollars per unit. This will lead to a very high gross profit margin for this product.
7) Selling precut lengths of stretch netting could also result in much higher gross profit margins for stretch netting sales when used in a Silent AC kit situation.

Finally, other advantages and additional features of the present non-kink IV device for placement on a patient's arm called a Silent antecubital or Silent AC will be more apparent from the accompanying drawings and from the full description of the device. For one skilled in the art of innovative medical accessories and components for improving patient care, it is readily understood that the features shown in the examples with this product can be adapted to other types of compact and hygienic medical systems, devices and accessories.

### DESCRIPTION OF THE DRAWINGS—FIGURES

The accompanying drawings, which are incorporated in and constitute a part of this specification, illustrate an embodiment of the non-kink IV device for placement on a patient's arm called a Silent antecubital or Silent AC. The drawings together with the summary description given above and a detailed description given below help to explain the principles of the Silent AC non-kink IV device. It is understood, however, that this Silent AC non-kink IV device is not limited to only the precise arrangements and instrumentalities shown.

FIGS. 1 A through 1 D are sketches portraying an overview of the non-kink IV device 30 and process 600 for placement on a patient's arm 105 called a Silent antecubital or Silent AC 30.

FIG. 2 is a typical IV on roll around carrier 81 with components and features noted.

FIGS. 3 A and 3 B are sketches of an intravenous (IV) device 37—intravenous usually refers to a way of giving a drug or other substance through a needle or tube inserted into a vein with the components and features shown from generally a top view.

FIGS. 4 A through 4 D are sketches of the option one type Silent antecubital or Silent AC of a roll of dressing gauze held by elastic straps, covered with a stretchy netting and with the components and features noted.

FIGS. 5 A through 5 E are sketches of the option two type Silent antecubital or Silent AC of a roll of dressing gauze, held by winged band aids, and covered with stretchy netting and with the components and features noted.

FIGS. 6 A through 6 D are sketches of the option three types of Silent antecubital or Silent AC with a roll of dressing gauze attached to the inner side of the stretchy netting and with the components and features noted.

FIGS. 7 A through 7 D are sketches of the option four type Silent antecubital or Silent AC with an inflatable bladder or soft rubber ball with a stretchy netting and with the components and features noted.

FIGS. 8 A through 8 F are sketches of prototypes of the option one type Silent antecubital or Silent AC of a roll of dressing gauze held by elastic straps, covered with a stretchy netting, showing the components and features noted.

FIGS. 9 A through 9 D are sketches that simulate the Silent AC directly attach to/couple with elastic bands for the prototypes of the option one type Silent antecubital or Silent AC of a roll of dressing gauze held by elastic straps, covered with a stretchy netting and with the components and features noted.

FIGS. 10 A through 10 D are sketches of the option four type Silent antecubital or Silent AC with an inflatable bladder or soft rubber ball with a stretchy netting and with the components and features noted.

FIG. 11 are sketches of the Process for Non-kink IV shown with option four type Silent antecubital or Silent AC with an inflatable bladder or soft rubber ball with a stretchy netting and with the components and features noted.

FIG. 12 A through 12 D are sketches of prior art.

### DESCRIPTION OF THE DRAWINGS—REFERENCE NUMERALS

The following list refers to the drawings:

TABLE B
Reference numbers -

| | |
|---|---|
| 30 | a non-kink IV device 30 for placement on a patient's arm called a |
| | Silent antecubital or Silent AC |
| 33 | an Intravenous (IV) end view 33 of the IV device 30 |
| 35 | a patient's arm - antecubital fossa 35 |
| 37 | an intravenous (IV) device 37 - intravenous usually refers to a way |
| | of giving a drug or other substance through a needle or tube |
| | inserted into a vein |
| 40 | a set of optional manners 40 to place the non-kink IV devices 30 |
| | for placement on a patient's arm 35 called a Silent antecubital or |
| | Silent AC |
| 41 | Option 1 41: compressible material 50 held by elastic bands 60 |
| | under another stretchy band 66 |
| 42 | Option 2 42: Silent AC 30 centered over IV insertion site with |
| | band aid like application Covered with transparent Dressing 88 |
| | Under Stretchy netting 66 to cover compressible material 50 and |
| | tubing 80 |
| 43 | Option 3 43: Silent AC 30 directly attached to Stretchy netting 66, |
| | centered over IV insertion site covered with transparent dressing |
| | 88 compressible material 50 and tubing 80 under stretchy netting |
| | 66 |
| 44 | an Option 4 44: Silent AC 30 centered over IV insertion site with |
| | an inflatable bladder 99 or soft rubber ball mass similar to the |
| | anchor system in a Foley Catheter |
| 50 | a compressible material 50 like a roll of gauze, a rubber ball |
| 51 | a rounded bottom 51 of the compressible material 50 like a roll |
| | of gauze |
| 60 | an elastic banding 60 |
| 66 | a stretchy netting 66 |
| 67 | a band aid attachment 67 - extended 67A; |
| 80 | a length of tubing 80 |
| 81 | a typical IV on roll around carrier 81 with elements identified |
| 85 | a cannula 85 |
| 86 | a clamp 86 |
| 87 | at least one Transparent tape 87 |
| 88 | a transparent tape 88 |
| 89 | a slider or roller clamp 89; |
| 90 | a port 90; a mainline 90A; a piggyback/secondary line 90B |
| 98 | a Luer lock port 98 for inflating with a syringe and air rubber |
| | ball |
| 99 | an inflatable bladder 99, a soft rubber “ball/mass” approximately |
| | 1 to 1½ inches in diameter and made with a fully FDA |
| | compliant material such as rubber, silicone, plastic such as |
| | nylon or urethane, polymethyl pentene (PMP), a polypropylene |
| | (PP), and polyurethane (PUR), a composite material, and a fiber |
| | cloth |
| 100 | a patient 100; |
| 105 | a nurse practitioner 105 |
| 600 | A process 600 for non-kink IV 30 |
| 700 | Prior Art 700 U.S. Pat. No. 5,112,313 Issued in 1987 to Sallee for |
| | an IV COVER/PROTECTOR |
| 710 | Prior Art 710 U.S. Pat. No. 4,976,698 Issued in 1990 to Stokley |
| | for an INTRAVENOUS CATHETER AND TUBING |
| | STABLIZATION DEVICE |
| 720 | Prior Art 720 U.S. Pat. No. 4,669,458 Issued in 1987 to Abraham |
| | et al. for an I.V. HOLDER |
| 730 | Prior Art 730 Pat App US 2006/0247577 A1 Submitted in 2006 by |
| | Wright for a FLEXIBLE IV SITE PROTECTOR |

### DETAILED DESCRIPTION OF PREFERRED EMBODIMENT

This invention relates to a non-kink IV device for placement on a patient's arm called a Silent antecubital or Silent AC. The present invention relates to a device for protecting an intravenous site of a patient from kinking and then setting off an alarm from a no-flow situation. Particularly, the embodiments described herein relate to devices and methods for transferring fluid to or from a patient through a placed peripheral intravenous catheter. The present invention generally relates to means of securing an intravenous catheter and tubing in use with a patient, and more particularly related to a device for securing and stabilizing an intravenous catheter and associated tubing adjacent to the site of entry of the catheter through the skin of the patient, protecting the catheter and its entry site from interference, and facilitating access to the catheter for maintenance by medical personnel while minimizing physical trauma to the patient.

The advantages for the non-kink IV device 30 for placement on a patient's arm called a Silent antecubital or Silent AC are listed above in the introduction. The benefits are that the system is shown for ADVANTAGES TO PATIENTS, ADVANTAGES TO NURSES, ADVANTAGES TO HOSPITALS and ADVANTAGES TO SUPPLIERS. These advantages are provided in Paragraphs [0013] through [0016] above.

The preferred embodiment of the Silent AC non-kink IV device 30 is comprised of: A non-kink IV device 30 called a Silent antecubital or Silent AC for placement on an intravenous (IV) device 37 on a patient's arm comprised of: (a) a compressed material such as a roll of gauze 50 with a rounded bottom 51; (b) At least one elastic band 60; and (c) a stretchy net piece to encase the roll and elastic bands Wherein the patient's Intravenous (IV) device 37 is held and the arm movement of the patient is limited to avoid kinking the tubing 80 and setting off an alarm. Other options of inflatable bladder/ball 99 and insert the roll into the stretchy netting 66 are also included.

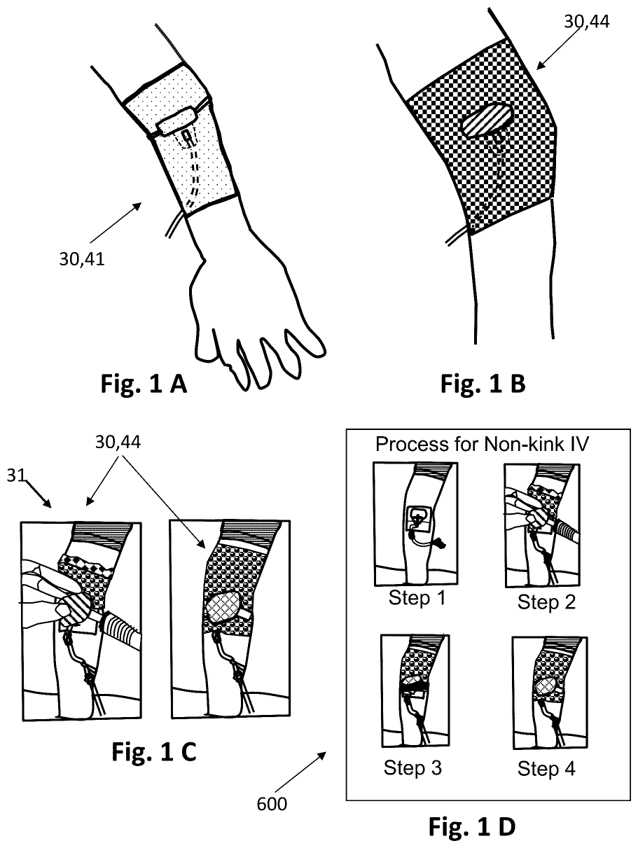

There is shown in FIGS. 1-12 a complete description and operative embodiment of the non-kink IV device called the Silent AC. In the drawings and illustrations, one notes well that FIGS. 1-12 demonstrate the general configuration and use of this product. The operation and use section below describe how it is installed with the patient. The accompanying drawings, which are incorporated in and constitute a part of this specification, illustrate an embodiment of the non-kink IV device 30. The drawings together with the summary description given above and a detailed description given below serve to explain the principles of the non-kink IV device 30 for placement on a patient's arm. It is understood, however, that device 30 is not limited to only the precise arrangements and instrumentalities shown. Other examples for using the device and their uses are still understood by one skilled in the art of medical devices and associated processes to be within the scope and spirit shown here.

FIGS. 1 A through 1 D are sketches portraying an overview of the non-kink IV device 30 and process 600 for placement on a patient's arm 105 called a Silent antecubital or Silent AC 30. Shown in this overview are: a non-kink IV device 30 for placement on a patient's arm called a Silent antecubital or Silent AC; an Option 1 41: compressible material 50 held by elastic bands 60 under stretchy netting 66; an Option 4 44: Silent AC 30 centered over IV insertion site with an inflatable bladder 99 or soft rubber ball mass Similar to the anchor system in a Foley Catheter; and a Process 600 for Non-kink IV 30.

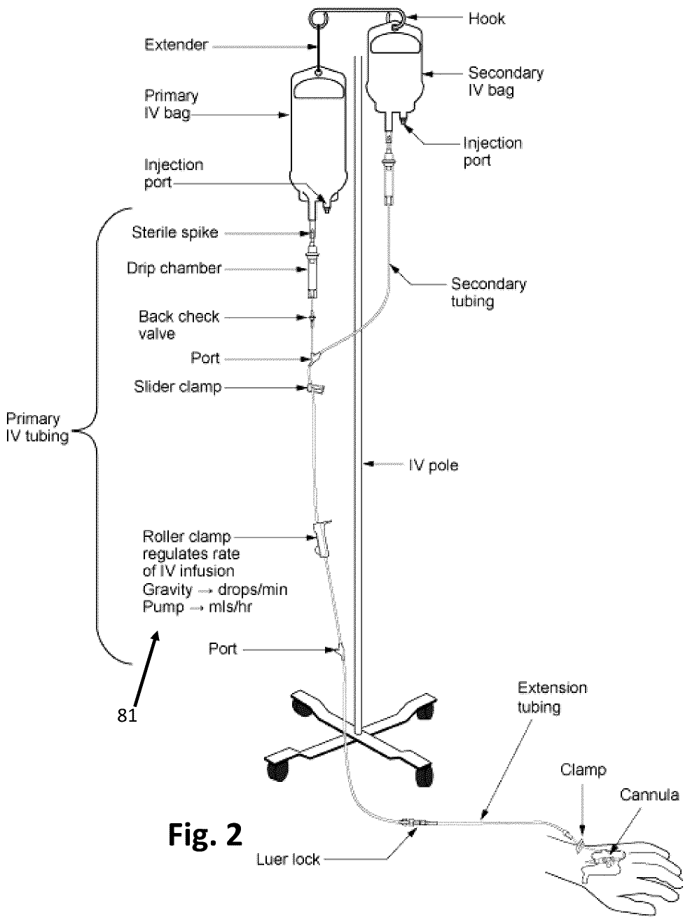

FIG. 2 is a typical IV on roll around carrier 81 with components and features noted. Portrayed here are those components in and around a patient's room that has an intravenous (IV) device 37 installed. Note that intravenous usually refers to a way of giving a drug or other substance through a needle or tube inserted into a vein.

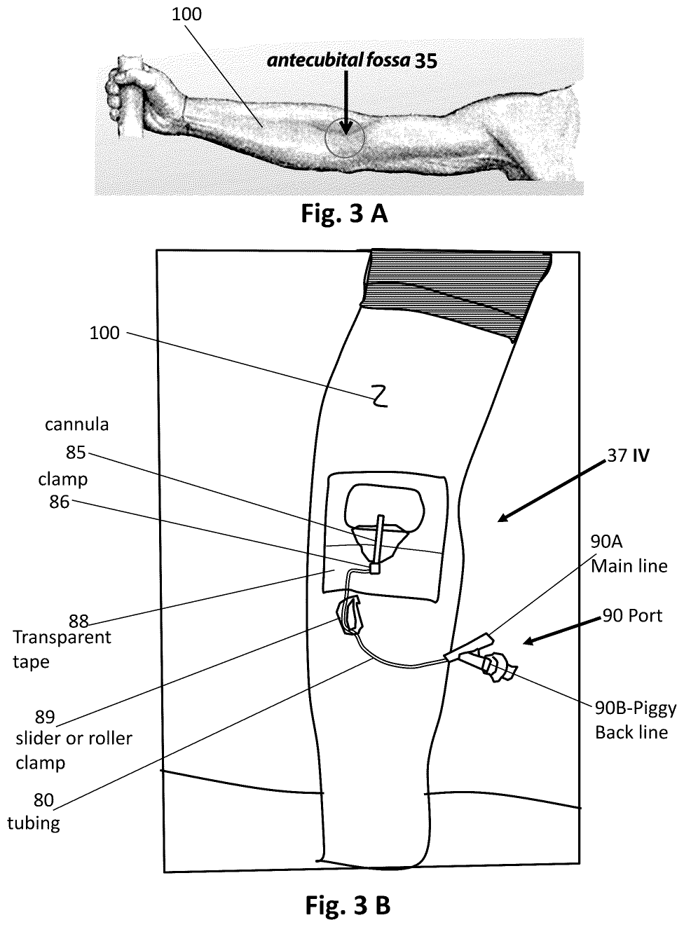

FIGS. 3 A and 3 B are sketches of an intravenous (IV) device 37 from generally a top view. Demonstrated here are: a patient's arm—antecubital fossa 35; an intravenous (IV) device 37 (intravenous usually refers to a way of giving a drug or other substance through a needle or tube inserted into a vein); a length of tubing 80; a cannula 85; a clamp 86; at least one Transparent tape 87; a transparent tape 88; a slider or roller clamp 89; a port 90; a mainline 90A; and a piggy back/secondary line 90B.

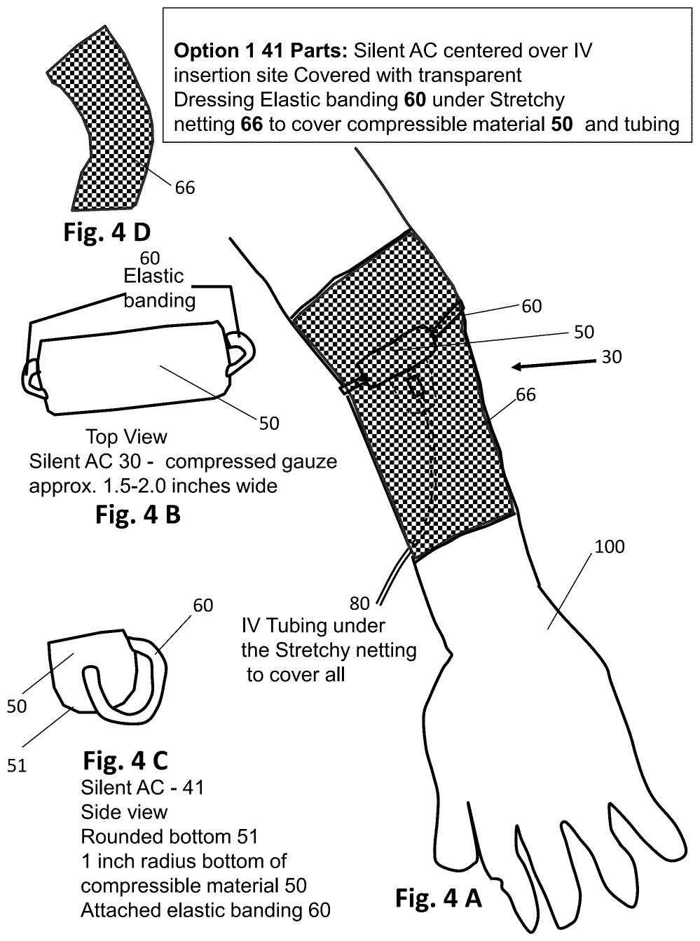

FIGS. 4 A through 4 D are sketches of the option one type Silent antecubital or Silent AC of a roll of dressing gauze held by elastic straps, covered with a stretchy netting and with the components and features noted. Shown in these sketches are: a non-kink IV device 30 for placement on a patient's arm called a Silent antecubital or Silent AC; a set of optional manners 40 to place the non-kink IV devices 30 for placement on a patient's arm 35 called a Silent antecubital or Silent AC; an Option 1 41: compressible material 50 held by elastic bands 60 under stretchy netting 66; a rounded bottom 51 of the compressible material 50 like a roll of gauze; a stretchy netting 66; a length of tubing 80; and a patient 100; a nurse practitioner 105; Process 600 for Non-kink IV 30.

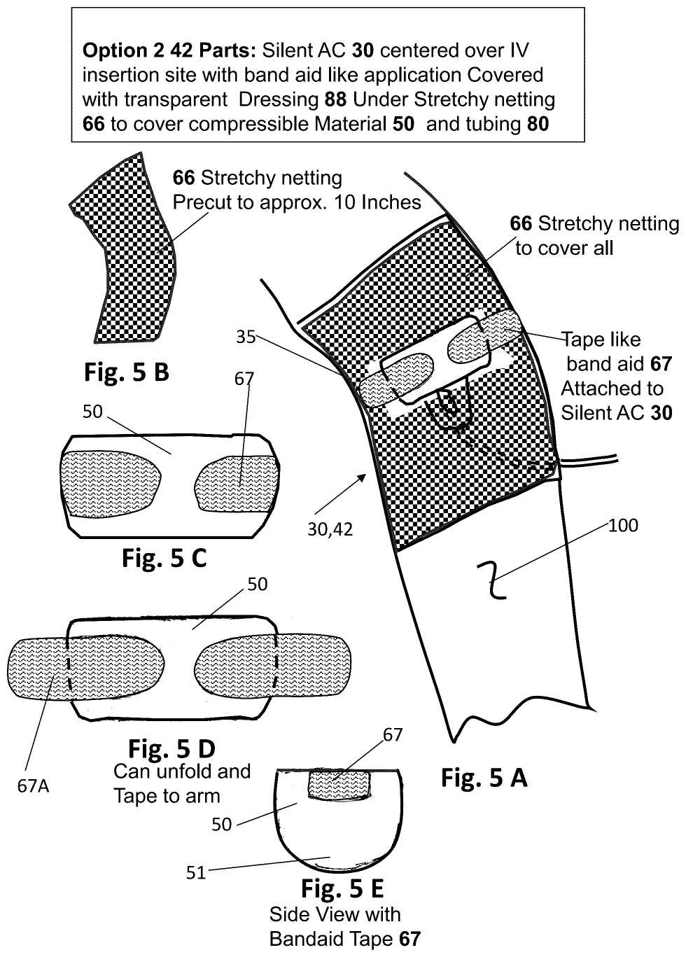

FIGS. 5 A through 5 E are sketches of the option two type Silent antecubital or Silent AC of a roll of dressing gauze, held by winged band aids, and covered with stretchy netting and with the components and features noted. Provided in Option 2 are: a non-kink IV device 30 for placement on a patient's arm called a Silent antecubital or Silent AC; an Option 2 42: Silent AC 30 centered over IV insertion site with band aid like application Covered with transparent Dressing 88 Under Stretchy netting 66 to cover compressible Material 50 and tubing 80; a stretchy netting 66; a band aid attachment 67—extended 67A; a length of tubing 80; and a patient 100.

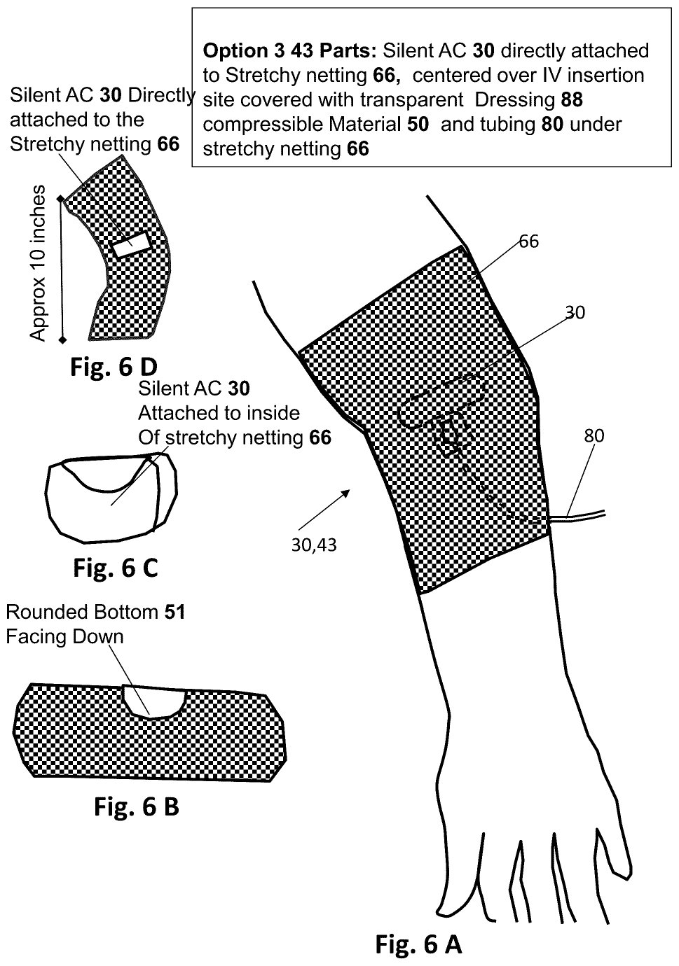

FIGS. 6 A through 6 D are sketches of the option three 43 type Silent antecubital or Silent AC with a roll of dressing gauze 50 attached to the inner side of the stretchy netting and with the components and features noted. Portrayed are: a non-kink IV device 30 for placement on a patient's arm called a Silent antecubital or Silent AC; an Option 3 43: Silent AC 30 directly attached to Stretchy netting 66, centered over IV insertion site covered with transparent Dressing 88 compressible Material 50 and tubing 80 under stretchy netting 66; a compressible material 50 like a roll of gauze; a rounded bottom 51 of the compressible material 50 like a roll of gauze; a stretchy netting 66; and a patient 100; a nurse practitioner 105; Process 600 for Non-kink IV 30.

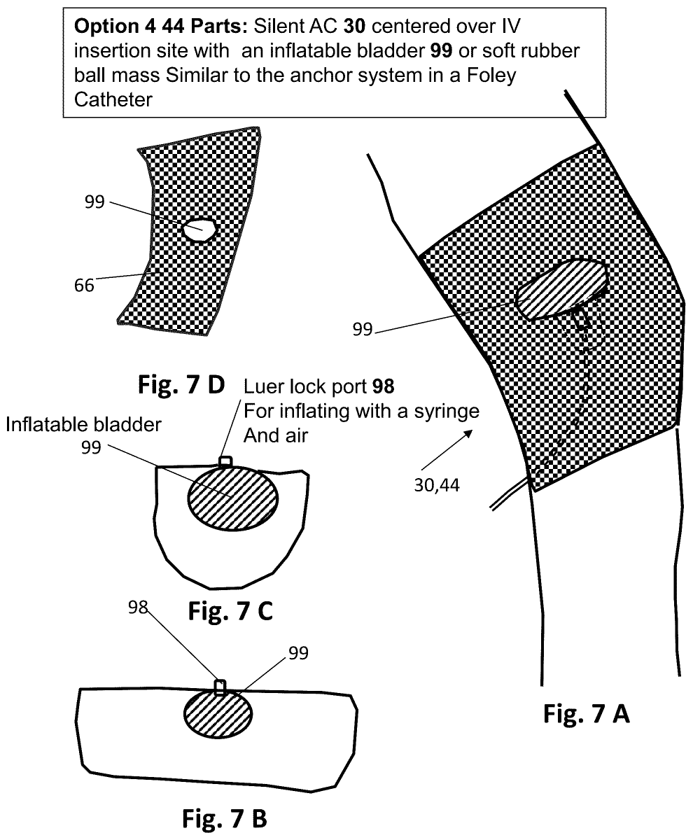

FIGS. 7 A through 7 D are sketches of the option four type Silent antecubital or Silent AC with an inflatable bladder 99 or soft rubber ball with a stretchy netting and with the components and features noted. The rubber ball 99 can be a soft rubber “ball/mass” or an inflatable bladder 99. The soft rubber “ball/mass” can be approximately 1 to 1½ inches in diameter and made with a fully FDA compliant material such as rubber, silicone, plastic such as nylon or urethane, Polymethyl pentene (PMP), a Polypropylene (PP), and Polyurethane (PUR), a composite material, and a fiber cloth. Presented in these sketches are: a non-kink IV device 30 for placement on a patient's arm called a Silent antecubital or Silent AC; an Option 4 44: Silent AC 30 centered over IV insertion site with an inflatable bladder 99 or soft rubber ball mass Similar to the anchor system in a Foley Catheter; a compressible material 50 like a roll of gauze; a stretchy netting 66; a length of tubing 80; a Luer lock port 98 for inflating with a syringe and air; an inflatable bladder 99 or soft rubber “ball/mass”; and a patient 100.

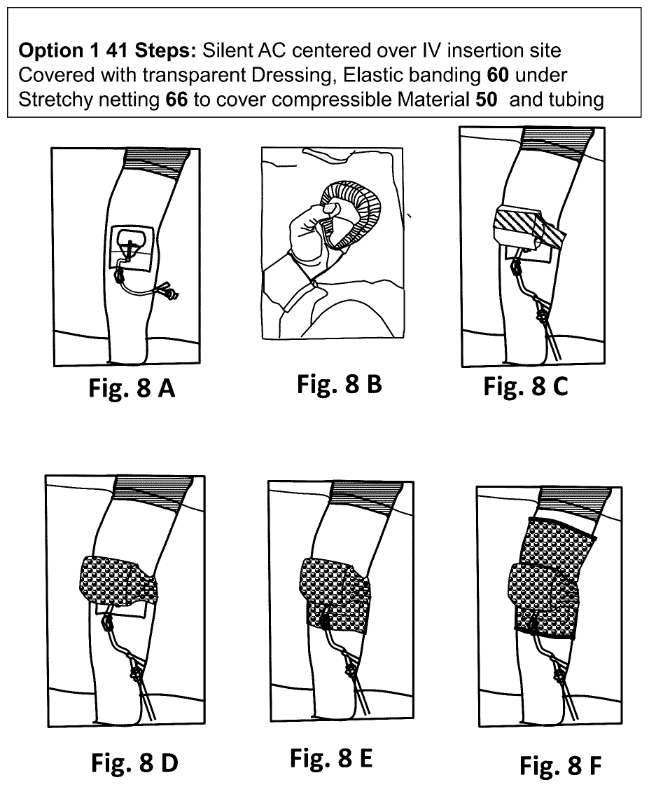

FIGS. 8 A through 8 F are sketches of prototypes of the option one 41 type Silent antecubital or Silent AC of a roll of dressing gauze held by elastic straps, covered with a stretchy netting, showing the components and features noted. Shown here are Option 1 41: compressible material 50 held by elastic bands 60 under stretchy netting 66; a patient 100; and a nurse practitioner 105.

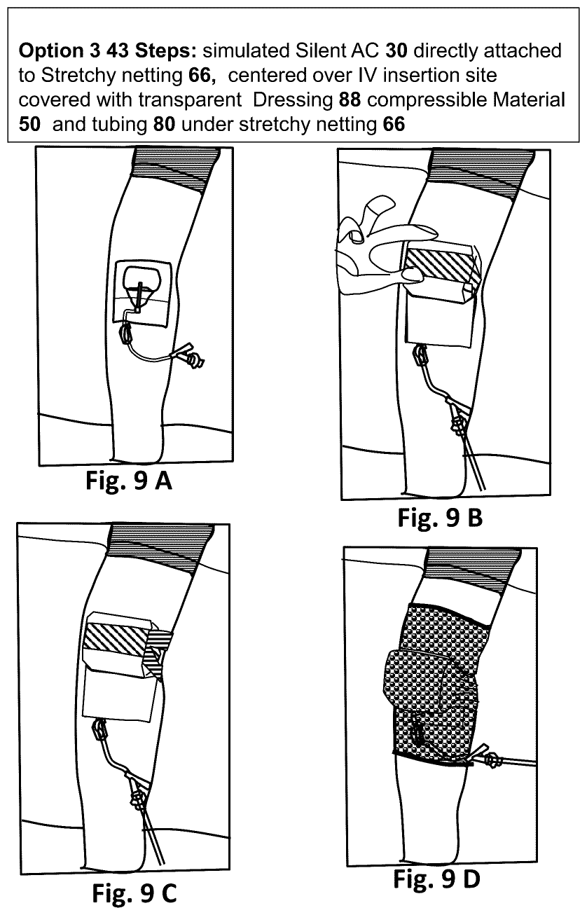

FIGS. 9 A through 9 D are sketches that simulate the Silent AC directly attach to/couple with elastic bands for the prototypes of the option one type Silent antecubital or Silent AC of a roll of dressing gauze held by elastic straps, covered with a stretchy netting and with the components and features noted. Here is provided: a non-kink IV device 30 for placement on a patient's arm called a Silent antecubital or Silent AC; Option 3 43: Silent AC 30 directly attached to Stretchy netting 66, centered over IV insertion site covered with transparent Dressing 88 compressible Material 50 and tubing 80 under stretchy netting 66; a patient 100; and a nurse practitioner 105.

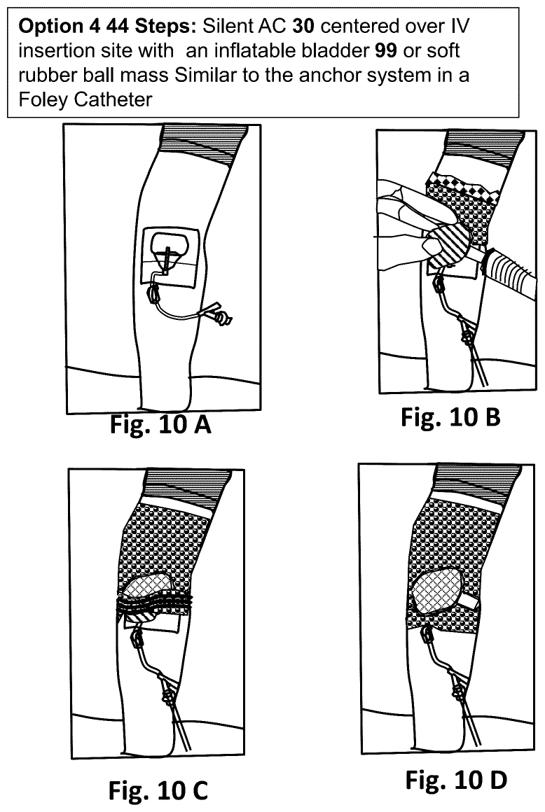

FIGS. 10 A through 10 D are sketches of the option four type Silent antecubital or Silent AC with an inflatable bladder 99 or soft rubber ball with a stretchy netting and with the components and features noted. Noted in these sketches are: a non-kink IV device 30 for placement on a patient's arm called a Silent antecubital or Silent AC; Option 4 44: Silent AC 30 centered over IV insertion site with an inflatable bladder 99 or soft rubber ball mass Similar to the anchor system in a Foley Catheter; a Luer lock port 98 for inflating with a syringe and air; an inflatable bladder 99 or soft rubber “ball/mass”; a patient 100; and a nurse practitioner 105.

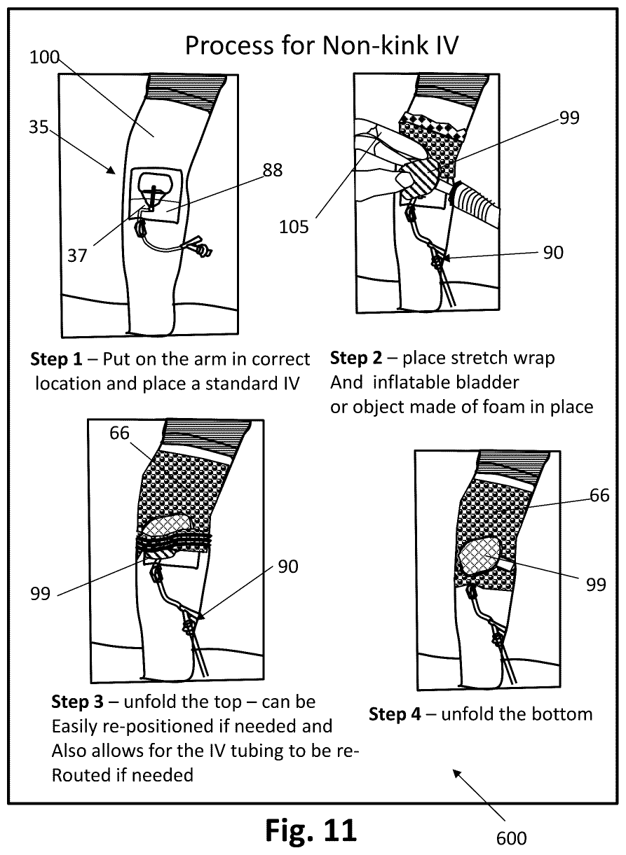

FIG. 11 are sketches of the Process 600 for Non-kink IV 30 shown with option four type Silent antecubital or Silent AC with an inflatable bladder 99 or soft rubber ball with a stretchy netting and with the components and features noted. This is described in the Operations Section below.

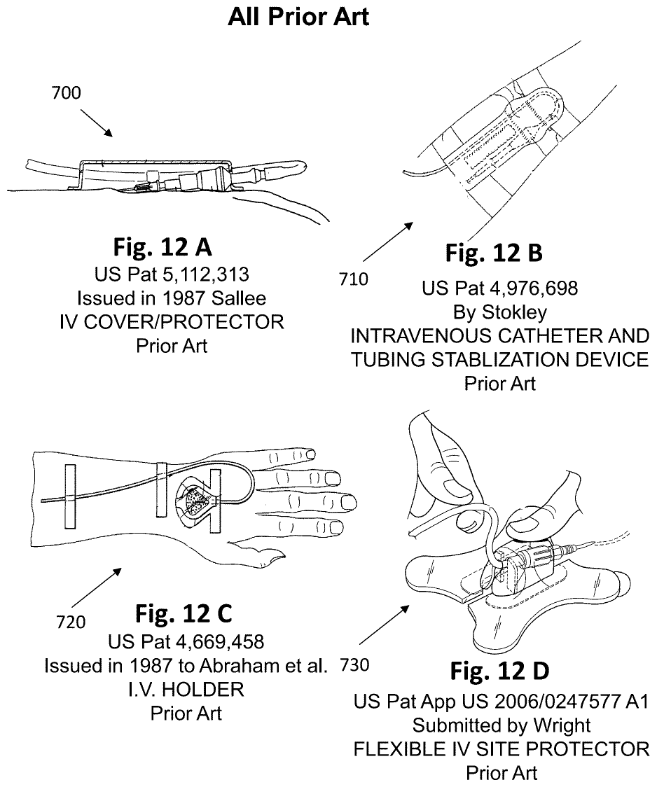

FIG. 12 A through 12 D are sketches of prior art. Here former patents and applications for medical devices incorporated with Intravenous systems are shown. These include: Prior Art 700 U.S. Pat. No. 5,112,313 Issued in 1987 to Sallee for an IV COVER/PROTECTOR; Prior Art 710 U.S. Pat. No. 4,976,698 Issued in 1990 to Stokley for an INTRAVENOUS CATHETER AND TUBING STABILIZATION DEVICE; Prior Art 720 U.S. Pat. No. 4,669,458 Issued in 1987 to Abraham et al. for an I.V. HOLDER; and Prior Art 730 Pat App US 2006/0247577 A1 Submitted in 2006 by Wright for a FLEXIBLE IV SITE PROTECTOR. As can be seen, the non-kink IV device 30 for placement on a patient's arm called a Silent antecubital or Silent AC is a unique medical device with the advantages shown above.

The materials and component shown for the non-kink IV device 30 for placement on a patient's arm are comprised of components that are hygienic and made for the safe, clean environment expected for the medical industry. called a Silent antecubital or Silent AC. The components will be made of materials that are medical grade or equal. These anticipated materials, for example and not limitation, include for the part contact surfaces—medical grade polymers and plastics that meet the stringent medical regulations. Medical-grade materials can include polystyrene, a common polymer. A medical-grade-plastic manufacturer's engineer the resins, with processes that enhance the qualities for biocompatibility. The results are polystyrene medical applications, from petri dishes to implants. A simple list for examples and not as a limitation includes PVC (both Flexible and Rigid), Polyethylene (ULDPE, LDPE, MDPE, HDPE), Polypropylene (homopolymer and copolymer), Polystyrene, Thermoplastic Elastomers (TPE's), Thermoplastic Polyurethane (TPU's)-Polyurethanes, Polycarbonate, and Nylon. Stainless steel or equal are used as well for some components.

The details mentioned here are exemplary and not limiting. Other specific components and manners specific to describing a Silent AC non-kink IV device 30 can be added as a person having ordinary skill in the field of the art of components and devices.

### OPERATION OF THE PREFERRED EMBODIMENT

The preferred embodiment of the Silent AC non-kink IV device 30 is comprised of: A non-kink IV device 30 called a Silent antecubital or Silent AC for placement on an intravenous (IV) device 37 on a patient's arm comprised of: (a) a compressed material such as a roll of gauze 50 with a rounded bottom 51; (b) At least one elastic band 60; and (c) a stretchy net piece to encase the roll and elastic bands Wherein the patient's Intravenous (IV) device 37 is held and the arm movement of the patient is limited to avoid kinking the tubing 80 and setting off an alarm. Other options of inflatable bladder/ball 99 and insert the roll into the stretchy netting 66 are also included.

SUCCESSFUL PLACEMENT OF THE SILENT AC DEVICE requires:
A. Silent AC must be placed directly proximally to the IV insertion site
B. Silent AC needs to have a rounded bottom that is on the patient's skin
C. Silent AC needs slight down pressure to help maintain patency of the IV (stretch netting provides the adequate down pressure required)

FIG. 11 are sketches of the Process 600 for Non-kink IV 30 shown with option four type Silent antecubital or Silent AC with an inflatable bladder 99 or soft rubber ball with a stretchy netting 66 and with the components and features noted. The process 600 used to install the non-kink IV device non-kink IV device 30 is as follows:
Step 1—Put on the arm in correct location and place a standard IV;
Step 2—place stretch wrap and inflatable bladder or object made of foam in place;
Step 3—unfold the top—can be Easily re-positioned if needed and allows for the IV tubing to be re-routed if needed;
Step 4—unfold the bottom; and
Step 5—remove and replace per instructions from the medical personnel 105.

With this description it is to be understood that the non-kink IV device 30 is not to be limited to only the disclosed embodiment of product. The features of the present improved medical device 30 are intended to cover various modifications and equivalent arrangements included within the spirit and scope of the description.

While certain novel features of this invention have been shown and described and are pointed out in the annexed claims, it is not intended to be limited to the details above, since it will be understood that various omissions, modifications, substitutions and changes in the forms and details of the device illustrated and in its operation can be made by those skilled in the art without departing in any way from the spirit of the present invention. Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

Unless defined otherwise, all technical and scientific terms used herein have the same meaning as commonly understood by one of the ordinary skills in the art to which these inventions belong. Although any methods and materials similar or equivalent to those described herein can also be used in the practice or testing of the present inventions, the preferred methods and materials are now described above in the foregoing paragraphs.

Other embodiments of the invention are possible. Although the description above contains much specificity, these should not be construed as limiting the scope of the invention, but as merely providing illustrations of some of the preferred embodiments of this invention. It is also contemplated that various combinations or sub-combinations of the specific features and aspects of the embodiments may be made and still fall within the scope of the inventions. It should be understood that various features and aspects of the disclosed embodiments can be combined with or substituted for one another in order to form varying modes of the disclosed inventions. Thus, it is intended that the scope of at least some of the present inventions herein disclosed should not be limited by the particular disclosed embodiments described above.

The terms recited in the claims should be given their ordinary and customary meaning as determined by reference to relevant entries (e.g., definition of “plane” as a carpenter's tool would not be relevant to the use of the term “plane” when used to refer to an airplane, etc.) in dictionaries (e.g., widely used general reference dictionaries and/or relevant technical dictionaries), commonly understood meanings by those in the art, etc., with the understanding that the broadest meaning imparted by any one or combination of these sources should be given to the claim terms (e.g., two or more relevant dictionary entries should be combined to provide the broadest meaning of the combination of entries, etc.) subject only to the following exceptions: (a) if a term is used herein in a manner more expansive than its ordinary and customary meaning, the term should be given its ordinary and customary meaning plus the additional expansive meaning, or (b) if a term has been explicitly defined to have a different meaning by reciting the term followed by the phrase “as used herein shall mean” or similar language (e.g., “herein this term means,” “as defined herein,” “for the purposes of this disclosure [the term] shall mean,” etc.). References to specific examples, use of “i.e.,” use of the word “invention,” etc., are not meant to invoke exception (b) or otherwise restrict the scope of the recited claim terms. Other than situations where exception (b) applies, nothing contained herein should be considered a disclaimer or disavowal of claim scope. Accordingly, the subject matter recited in the claims is not coextensive with and should not be interpreted as coextensive with any particular embodiment, feature, or combination of features shown herein. This is true even if only a single embodiment of the particular feature or combination of features is illustrated and described herein. Thus, the appended claims should be read to be given their broadest interpretation in view of the prior art and the ordinary meaning of the claim terms.

Unless otherwise indicated, all numbers or expressions, such as those expressing dimensions, physical characteristics, etc. used in the specification (other than the claims) are understood as modified in all instances by the term “approximately.” At the very least, and not as an attempt to limit the application of the doctrine of equivalents to the claims, each numerical parameter recited in the specification or claims which is modified by the term “approximately” should at least be construed in light of the number of recited significant digits and by applying ordinary rounding techniques.

The present invention contemplates modifications as would occur to those skilled in the art. While the disclosure has been illustrated and described in detail in the figures and the foregoing description, the same is to be considered as illustrative and not restrictive in character, it being understood that only selected embodiments have been shown and described and that all changes, modifications and equivalents that come within the spirit of the disclosures described heretofore and or/defined by the following claims are desired to be protected.

What is claimed is:

1. A non-kink IV device (30) called a Silent antecubital (AC) for placement onto an intravenous (IV) device (37) on a patient's arm, the device (37) comprised of:
(a) a compressed material (50) with a rounded bottom (51);
(b) At least one elastic banding (60); and
(c) A stretchy netting (66) piece to encase the compressed material (50) and elastic banding (66)
wherein the patient's Intravenous (IV) device (37) is held, and the arm movement of the patient is limited to avoid kinking the tubing (80) and setting off an alarm.

2. A non-kink IV device (30) described in claim 1 wherein the compressible material (50) is an inflatable bladder/ball (99).

3. A non-kink IV device (30) described in claim 1 wherein the compressible material (50) is a soft “ball/mass” approximately 1 to 1½ inches in diameter and made with a fully FDA compliant material.

4. A non-kink IV device (30) described in claim 3 wherein the fully FDA compliant material is selected from a group consisting of a rubber, a silicone, a plastic, a composite material, and a fiber cloth.

5. A non-kink IV device (30) described in claim 4 wherein the plastic is selected from a group consisting of a nylon, a urethane, a Polymethyl pentene (PMP), a Polypropylene (PP), and a Polyurethane (PUR).

6. A non-kink IV device (30) called a Silent antecubital (AC) for placement onto an intravenous (IV) device (37) on a patient's arm, the device (37) comprised of:
(a) a compressed material (50) with a rounded bottom (51);
(b) At least one elastic banding (60);
(c) A stretchy netting (66) piece to encase the compressed material (50) and elastic banding (60); and
(d) a band aid (67) application covered with a transparent tape (88) under stretchy netting 66 to cover the compressible material (50) and any IV tubing (80)
wherein the patient's Intravenous (IV) device (37) is held, and the arm movement of the patient is limited to avoid kinking the tubing (80) and setting off an alarm.

7. A non-kink IV device (30) described in claim 6 wherein the compressible material (50) is an inflatable bladder/ball (99).

8. A non-kink IV device (30) described in claim 6 wherein the compressible material (50) is a soft “ball/mass” approximately 1 to 1½ inches in diameter and made with a fully FDA compliant material.

9. A non-kink IV device (30) described in claim 8 wherein the fully FDA compliant material is selected from a group consisting of a rubber, a silicone, a plastic, a composite material, and a fiber cloth.

10. A non-kink IV device (30) described in claim 9 wherein the plastic is selected from a group consisting of a nylon, a urethane, a Polymethyl pentene (PMP), a Polypropylene (PP), and a Polyurethane (PUR).

11. A process (600) used to install a non-kink IV device (30) is as follows:
Step 1—Put device (30) on the arm in correct location and place a standard IV;
Step 2—place stretch wrap and a compressible material (50) in place over the IV;
Step 3—unfold the top and re-positioned if needed and allows for the IV tubing to be re-routed if needed;
Step 4—unfold the bottom; and
Step 5—remove and replace per instructions from the medical personnel (105).
